(19) United States
(12) Patent Application Publication
(10) Pub. No.: US 2018/0271499 A1
(43) Pub. Date: Sep. 27, 2018

(54) INFORMATION PROCESSING DEVICE AND INFORMATION PROCESSING METHOD

(71) Applicant: SONY CORPORATION, TOKYO (JP)

(72) Inventor: YOICHI TORIUMI, TOKYO (JP)

(73) Assignee: Sony Corporation, Tokyo (JP)

(21) Appl. No.: 15/760,339

(22) PCT Filed: Sep. 15, 2016
(86) PCT No.: PCT/JP2016/077224

§ 371 Date: Mar. 15, 2018

(30) Foreign Application Priority Data
Sep. 29, 2015 (JP) 2015-190831

(51) Int. Cl. A61B 8/00; A61B 8/14; A61B 5/00; G06F 1/26; H04W 12/06

(52) U.S. Cl. CPC A61B 8/565; A61B 8/14; A61B 8/4236; H04W 84/18; G06F 1/26; H04W 12/06; A61B 5/0066

(57) ABSTRACT

The present technology relates to an information processing device and an information processing method that enable the convenience of a sensor unit to improve. The information processing device according to the present technology includes: an authentication unit configured to perform authentication processing with a different information processing device; and a power supply unit configured to supply power to the different information processing device with which the authentication processing has been performed, in a case where authentication of the authentication unit succeeds. The present technology can be applied to, for example, an information processing device, a biological sensor unit, a radio cooperative apparatus, a power supply device, or a radio communication device.

### TECHNICAL FIELD

The present technology relates to an information processing device and an information processing method, and particularly relates to an information processing device and an information processing method, capable of improving the convenience of a sensor unit.

### BACKGROUND ART

Conventionally, there is a system in which a patch-type sensor unit is applied to the skin of a bedridden patient and a radio cooperative apparatus brought close from above the clothes, acquires, in radio communication, a biological signal of the bedridden patient detected by the sensor unit and analyzes the biological signal.

In addition, for example, there are a patch-type sensor unit having a function of detecting biological information with an ultrasonic echo, the patch-type sensor being configured to sense a defecation desire after 10 minutes from detected biological information, being applied to the skin in the vicinity of a rectum, and a smartphone application that issues a notification of the defecation desire in cooperation with the sensor unit.

Furthermore, there is a patch including a sensor module and a RFID tag, the patch being configured to drive with reception of only feeble radio electric supply approximate to a degree of electromagnetic induction with which the RFID tag drives, from a mobile phone (e.g., refer to Patent Document 1).

### CITATION LIST

### Patent Document

Patent Document 1: Japanese Unexamined Patent Application Publication (Translation of PCT Application) No. 2007-519484

### SUMMARY OF THE INVENTION

### Problems to be Solved by the Invention

However, the feeble electric supply for the patch described in Patent Document 1 allows only limited sensor modules to drive. For example, a sophisticated sensor for ultrasonic echo measurement is difficult to drive, and a high degree of biological information is difficult to acquire.

The conventional sensor units each have a battery as a power source and perform signal processing in order to perform a high degree of sensing, such as the ultrasonic echo measurement. Thus, for example, a casing is too large to apply such a sensor unit at all times, substantially interfering with life, and thus there is a risk that the convenience of the sensor unit decreases.

The present technology has been proposed in consideration of the situations, and an object of the present technology is to improve the convenience of a sensor unit.

### Solutions to Problems

An information processing device according to a first aspect of the present technology, includes: an authentication unit configured to perform authentication processing with a different information processing device; and a power supply unit configured to supply power to the different information processing device in a case where authentication of the authentication unit succeeds.

The power supply unit can supply preliminary power for the authentication to the different information processing device before the authentication of the authentication unit, the power supply unit being able to start supplying normal power for driving the different information processing device in a normal state, to the different information processing device in the case where the authentication of the authentication unit succeeds, the normal power having power larger than the preliminary power, the power supply unit being able to stop supplying the power in a case where the authentication of the authentication unit fails.

The power supply unit can supply the power to the different information processing device in radio communication.

The authentication unit and the different information processing device can authenticate each other.

A reception unit configured to receive a transmitted signal transmitted by the different information processing device, can be further provided.

The transmitted signal can be made a radio signal.

The transmitted signal can include sensor information being information detected by a sensor included in the different information processing device.

The power supply unit can finish the supply of the power after the reception unit receives the sensor information.

An information processing unit configured to process the sensor information received by the reception unit, can be further provided.

An information processing method according to the first aspect of the present technology, includes: performing authentication processing with a different information processing device; and supplying power to the different information processing device in a case where authentication succeeds.

An information processing device according to a second aspect of the present technology, includes: an authentication unit configured to perform authentication processing with a different information processing device; and a power acquisition unit configured to acquire power supplied from the different information processing device in a case where authentication of the authentication unit succeeds.

The power acquisition unit can acquire the power supplied from the different information processing device in radio communication.

The authentication unit and the different information processing device can authenticate each other.

The power acquisition unit can acquire preliminary power for the authentication, supplied from the different information processing device before the authentication of the authentication unit. The authentication unit can perform the authentication processing with the preliminary power acquired by the power acquisition unit. The power acquisition unit can start acquiring normal power for driving the information processing device in a normal state, supplied from the different information processing device in the case where the authentication of the authentication unit succeeds, the normal power having power larger than the preliminary power, the power acquisition unit being able to prohibit the acquisition of the power in a case where the authentication of the authentication unit fails.

A transmission unit configured to transmit a transmitted signal to the different information processing device, can be further provided, the transmission unit being configured to drive with the normal power acquired by the power acquisition unit.

The transmitted signal can be made a radio signal.

A sensor unit configured to detect a predetermined parameter, can be further provided, the sensor unit being configured to drive with the normal power acquired by the power acquisition unit. The transmission unit can transmit sensor information being information regarding a detected result of the sensor unit, as the transmitted signal.

The sensor unit can detect biological information being information regarding a living body.

The power acquisition unit can finish the acquisition of the normal power after the transmission unit finishes the transmission of the transmitted signal.

An information processing method according to the second aspect of the present technology, includes: performing authentication processing with a different information processing device; and acquiring power supplied from the different information processing device in a case where authentication succeeds.

In the information processing device and the information processing method according to the first aspect of the present technology, the authentication processing is performed with the different information processing device, and the power is supplied to the different information processing device in the case where the authentication succeeds.

In the information processing device and the information processing method according to the second aspect of the present technology, the authentication processing is performed with the different information processing device, and the power supplied from the different information processing device is acquired in the case where the authentication succeeds.

### Effects of the Invention

According to the present technology, measurement with light can be performed. In addition, according to the present technology, the convenience of the sensor unit can improve.

### BRIEF DESCRIPTION OF DRAWINGS

FIG. 1 is a block diagram illustrating exemplary configurations of a conventional sensor unit and radio cooperative apparatus.

FIG. 2 is a block diagram illustrating exemplary configurations of a sensor unit and a radio cooperative apparatus.

FIG. 3 is a diagram for describing exemplary utilization.

FIG. 4 is a flowchart for describing an exemplary flow of detection processing.

FIG. 5 is a flowchart for describing an exemplary flow of authentication processing.

FIG. 6 is a flowchart for describing an exemplary flow of authentication processing.

FIG. 7 is a diagram for describing exemplary utilization of biological information.

FIG. 8 is a diagram for describing exemplary utilization of the biological information.

FIG. 9 is a block diagram illustrating an exemplary main configuration of a computer.

### MODE FOR CARRYING OUT THE INVENTION

A mode for carrying out the present disclosure (hereinafter, referred to as an embodiment) will be described below. Note that the descriptions will be given in the following order.

### 1. First Embodiment (Sensor System)

### 1. First Embodiment

<Power Supply>

Conventionally, there is a sensor system in which a patch-type sensor unit is applied to the skin of a bedridden patient and a radio cooperative apparatus brought close from above the clothes, acquires, in radio communication, a biological signal of the bedridden patient detected by the sensor unit and analyzes the biological signal.

In addition, for example, there are a patch-type sensor unit having a function of detecting biological information with an ultrasonic echo, the patch-type sensor being configured to sense a defecation desire after 10 minutes from detected biological information, being applied to the skin in the vicinity of a rectum, and a smartphone application that issues a notification of the defecation desire in cooperation with the sensor unit.

However, the sensor units each have a battery as a power source and perform signal processing in order to perform a high degree of sensing, such as ultrasonic echo measurement.

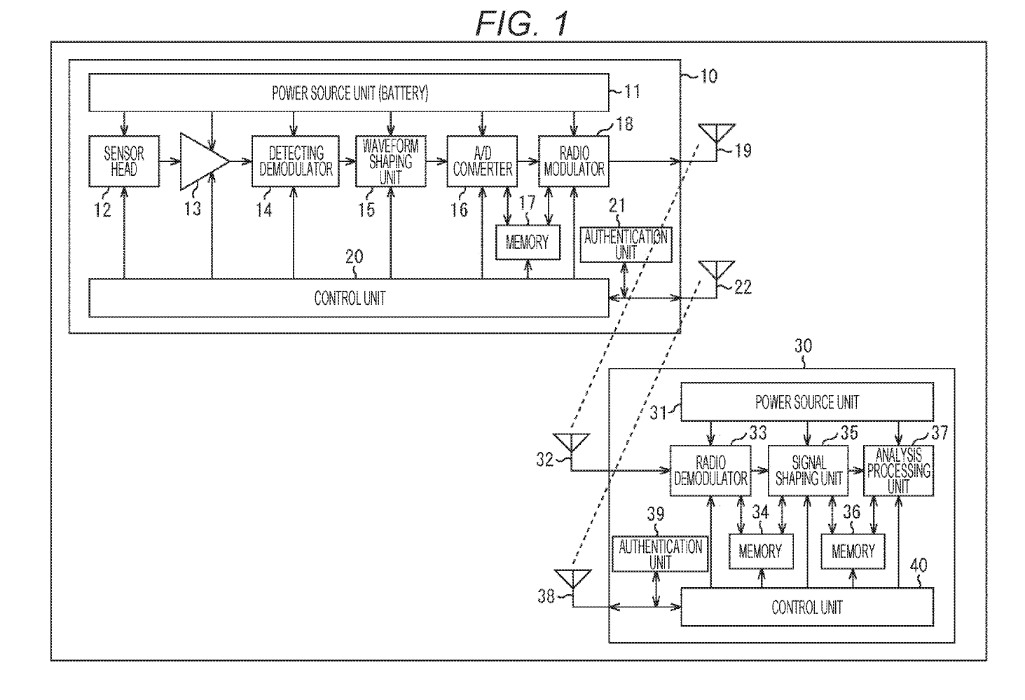

FIG. 1 is a block diagram illustrating exemplary main configurations of a conventional sensor unit and radio cooperative apparatus. As illustrated in FIG. 1, the conventional sensor unit 10 includes a power source unit (a battery) 11, a sensor head 12, an amplifier 13, a detecting demodulator 14, a waveform forming unit 15, an A/D converter 16, a memory 17, a radio modulator 18, an antenna 19, a control unit 20, an authentication unit 21, and an antenna 22.

Biological information detected by the sensor head 12 is amplified by the amplifier 13. The detecting demodulator 14 and the waveform forming unit 15 each perform signal processing to the biological information. The A/D converter 16 performs A/D conversion to the biological information. The memory 17 stores the biological information. When radio communication with the radio cooperative apparatus 30 is allowed, the radio modulator 18 reads and modulates the biological information from the memory 17 and transmits the biological information as a radio signal from the antenna 19.

The processing units drive with reception of power supply from the power source unit (the battery) 11. In addition, the control unit 20 controls the processing units to drive. The authentication unit 21 authenticates the radio cooperative apparatus 30 in radio communication with the radio cooperative apparatus 30 through the antenna 22. When the authentication unit 21 authenticates the radio cooperative apparatus 30, the control unit 20 controls each of the processing units such that the biological information is supplied to the radio cooperative apparatus 30 as described above.

In addition, the conventional radio cooperative apparatus 30 includes a power source unit 31, an antenna 32, a radio demodulator 33, a memory 34, a signal shaping unit 35, a memory 36, an analysis processing unit 37, an antenna 38, an authentication unit 39, and a control unit 40.

The radio signal transmitted from the sensor unit 10 is received by the radio demodulator 33 through the antenna 32. The radio demodulator 33 demodulates the received signal to extract the biological information, and stores the biological information into the memory 34. The signal shaping unit 35 acquires the biological information from the memory 34, shapes a signal of the biological information, and stores the signal into the memory 36. The analysis processing unit 37 acquires the biological information from the memory 36 to perform processing of, for example, analyzing the biological information and outputting an analyzed result.

The processing units drive with reception of power supply from the power source unit 31. In addition, the control unit 40 controls the processing units to drive. The authentication unit 39 performs authentication processing in radio communication with the sensor unit 10 through the antenna 38. When the authentication succeeds, the control unit 40 controls each of the processing units such that the biological information is acquired and analyzed as described above.

The battery is required as the power source and the various types of processing are performed in the conventional sensor unit 10 in this manner. For example, a casing is too large to apply the sensor unit at all times, substantially interfering with life, and thus there is a risk that the convenience of the sensor unit decreases.

Therefore, a radio cooperative apparatus performs authentication processing with a different information processing device, and supplies power to the different information processing device in a case where the authentication succeeds. That is, the radio cooperative apparatus includes: an authentication unit that performs the authentication processing with the different information processing device; and a power supply unit that supplies the power to the different information processing device in the case where the authentication of the authentication unit succeeds.

In addition, a sensor unit performs authentication processing with a different information processing device, and acquires power supplied from the different information processing device in a case where the authentication succeeds. That is, the sensor unit includes: an authentication unit that performs the authentication processing with the different information processing device; and a power acquisition unit that acquires the power supplied from the different information processing device in the case where the authentication of the authentication unit succeeds.

<Sensor System>

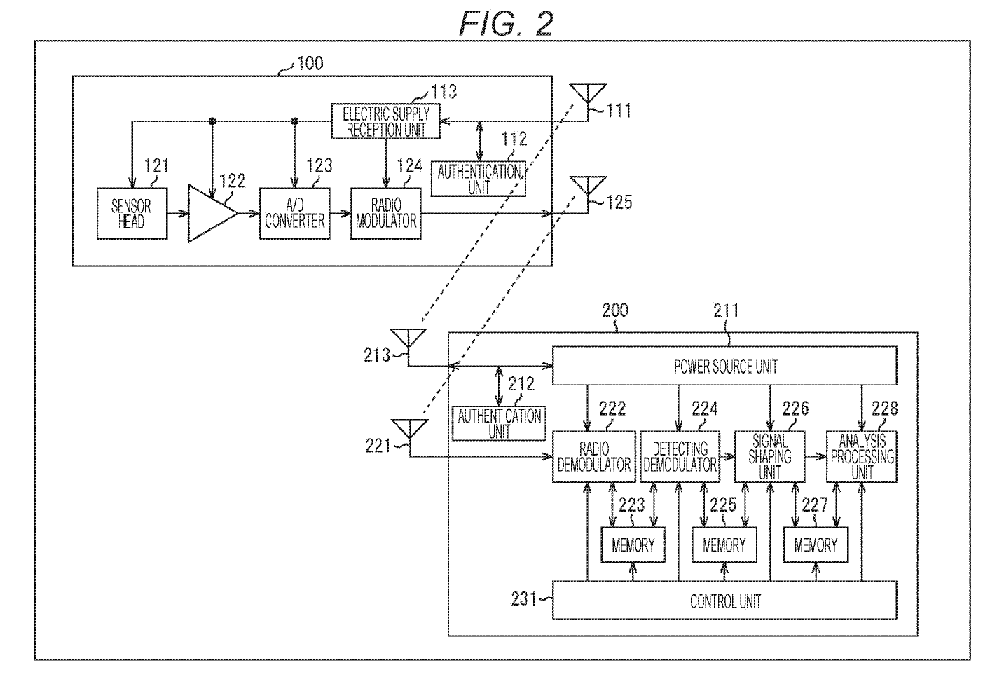

A more specific description will be given. FIG. 2 is a block diagram illustrating an exemplary sensor system to which the present technology has been applied. The sensor system includes a biological sensor unit 100 and a radio cooperative apparatus 200, and is a system in which the radio cooperative apparatus 200 acquires, in radio communication, and analyzes biological information being information regarding a living body detected by the biological sensor unit 100.

The biological sensor unit 100 detects biological information regarding a user (a human body), the biological sensor unit 100 being applied to the skin of the user. The radio cooperative apparatus 200 acquires and processes the biological information detected by the biological sensor unit 100 in radio communication with the biological sensor unit 100. In addition, the radio cooperative apparatus 200 supplies power to the biological sensor unit 100 in radio communication.

As illustrated in FIG. 2, the biological sensor unit 100 includes an antenna 111, an authentication unit 112, an electric supply reception unit 113, a sensor head 121, an amplifier 122, an A/D converter 123, a radio modulator 124, and an antenna 125.

The electric supply reception unit 113 receives electric supply in a radio mode (radio electric supply) from the radio cooperative apparatus 200 through the antenna 111, and supplies the power to the sensor head 121 to the radio modulator 124 to drive.

For the radio electric supply, the authentication unit 112 performs authentication processing in radio communication with the radio cooperative apparatus 200 through the antenna 111. In a case where the authentication succeeds, the supply of the power for driving the sensor head 121 to the radio modulator 124 in a normal state (hereinafter, also referred to as normal power), from the radio cooperative apparatus 200, is allowed. In a case where the authentication fails, the supply of the normal power is prohibited.

The sensor head 121 has an arbitrary sensor that detects an arbitrary parameter, and performs processing regarding the detection of the biological information. That is, the sensor including in the sensor head 121 may be any sensor, and the sensor may detect any information as the biological information. That is, the contents of the biological information are also arbitrary. For example, the sensor head 121 may include an ultrasonic echo sensor that applies an ultrasonic wave to an object and brings the reflection thereof into visualization to examine a state inside the object, or an optical coherence tomography (OCT) sensor that irradiates light (e.g., a laser beam) and detects the reflected light thereof to examine a state inside an object. Such sensors perform a high degree of processing in which, for example, light or an ultrasonic wave is irradiated and the reflection thereof is detected, and thus power is required larger than power approximately to a degree of feeble electromagnetic induction with which a RFID tag drives.

The biological information detected by the sensor head 121 is amplified by the amplifier 122. The A/D converter 123 performs A/D conversion to the biological information. The radio modulator 124 modulates the biological information in a predetermined modulation mode and transmits the biological information as a radio signal from the antenna 125. In this case, the sensor head 121 to the radio modulator 124 drive with the power supplied, namely, communication with the radio cooperative apparatus 200 can be performed. Therefore, the biological information detected by the sensor head 121 can be transmitted without being retained in a memory.

In addition, the radio cooperative apparatus 200 includes a power source unit 211, an authentication unit 212, an antenna 213, an antenna 221, a radio demodulator 222, a memory 223, a detecting demodulator 224, a memory 225, a signal shaping unit 226, a memory 227, an analysis processing unit 228, and a control unit 231.

The power source unit 211 supplies the power to each of the processing units in the radio cooperative apparatus 200, and additionally performs the radio electric supply to the biological sensor unit 100 through the antenna 213. The mode of the radio electric supply is arbitrary, and may be, for example, an electromagnetic induction mode or a radio wave mode. The power source unit 211 can supply the normal power larger than the power for driving the RFID tag, to the biological sensor unit 100 with the radio electric supply.

For the radio electric supply, the authentication unit 212 performs authentication processing in radio communication with the biological sensor unit 100 through the antenna 111. In a case where the authentication succeeds, the supply of the normal power to the biological sensor unit 100 is allowed. In a case where the authentication fails, the supply of the normal power is prohibited.

The radio signal transmitted from the biological sensor unit 100 is received by the radio demodulator 222 through the antenna 221. The radio demodulator 33 demodulates the received signal in a predetermined demodulation mode to extract the biological information, and stores the biological information into the memory 223. The detecting demodulator 224 acquires the biological information from the memory 223, detects and demodulates the biological information in a predetermined mode to store the biological information into the memory 225. The signal shaping unit 226 acquires the biological information from the memory 225, and shapes a signal of the biological information to store the signal into the memory 227. The analysis processing unit 228 acquires the biological information from the memory 227 to perform processing of, for example, analyzing the biological information and outputting an analyzed result. The control unit 231 controls the processing units to drive.

<Exemplary Utilization>

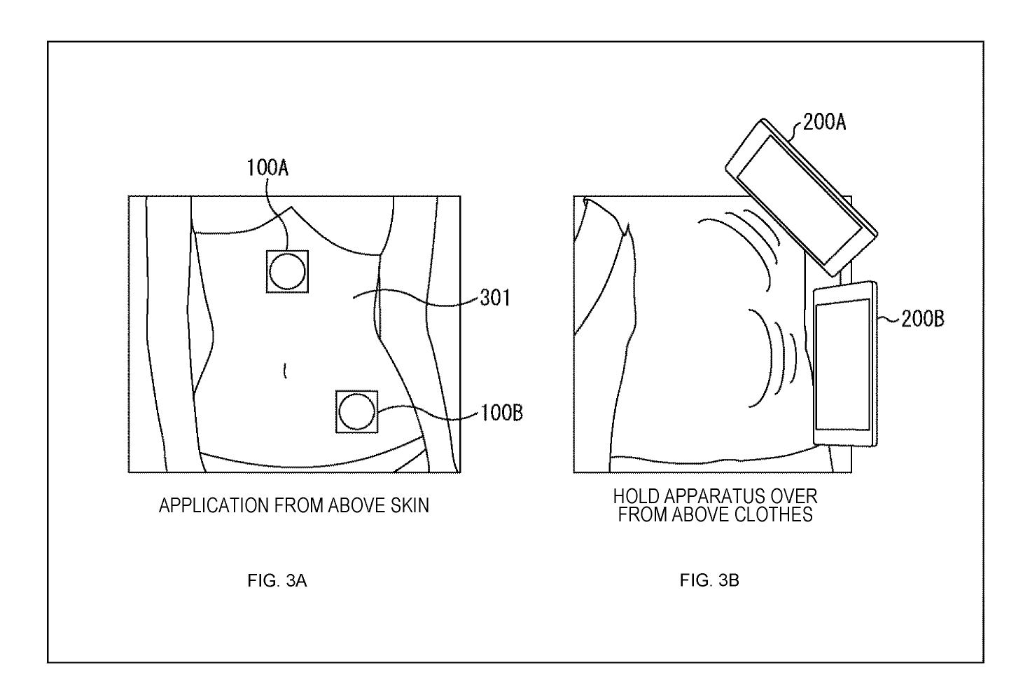

For example, the biological sensor unit 100 is used with a patch, a label, or the like as in A of FIG. 3, the biological sensor unit 100 being applied to the skin of a user (a human body) 301 as an object to be detected for biological information. For example, a biological sensor unit 100A is applied in the vicinity of the stomach of the user 301. In addition, for example, a biological sensor unit 100B is applied in the vicinity of a intestine of the user 301.

For example, as in B of FIG. 3, when a user (not necessarily the user 301 applied with the biological sensor unit 100) holds the radio cooperative apparatus 200 over the biological sensor unit 100 (approach to a distance at which the radio electric supply and the radio communication can be performed to the biological sensor unit 100), the biological sensor unit 100 drives to perform sensing of the biological information and transmits information regarding the sensor result thereof (sensor information), to the radio cooperative apparatus 200. For example, the biological sensor unit 100 performs ultrasonic echo examination or OCT examination, detects information regarding a reflected wave (namely, information indicating the condition of the stomach or the intestine), and transmits the information regarding the reflected wave as the sensor information (the biological information) to the radio cooperative apparatus 200.

For example, the radio cooperative apparatus 200A is held over the biological sensor unit 100A applied to the skin, from above the clothes. In this case, the biological sensor unit 100A transmits, for example, the echo diagnosis result of the stomach and the like as the biological information to the radio cooperative apparatus 200A. In addition, for example, the radio cooperative apparatus 200B is held over the biological sensor unit 100B applied to the skin, from above the clothes. In this case, the biological sensor unit 100B transmits, for example, the echo diagnosis result of the intestine and the like as the biological information to the radio cooperative apparatus 200B.

As illustrated in FIG. 2, the biological sensor unit 100 has no power source (no battery), and thus drives with the electric supply from the radio cooperative apparatus 200. In addition, the biological sensor unit 100 performs only the signal processing necessary for transmitting, as the radio signal, the biological information acquired by the sensor head 121, and the radio cooperative apparatus 200 performs the signal processing performable in the radio cooperative apparatus 200. Therefore, miniaturization of the biological sensor unit 100 can be achieved more easily. That is, the biological sensor unit 100 can be made smaller, thinner, and softer.

Thus, therefore, even when the biological sensor unit 100 is applied to the skin of the user at all times, influence exerted on the user's life can be reduced. Therefore, there is no need to apply and remove the biological sensor unit 100 in every piece of measurement, and thus the measurement can be performed more easily. With this arrangement, the sensor system (the biological sensor unit 100 and the radio cooperative apparatus 200) can be used not only for examination of a bedridden patient but also for measurement in an able-bodied person's daily life. That is, the convenience of the biological sensor unit 100 can improve.

In addition, since the biological sensor unit 100 has no power source (no battery) as described above, the power supply to the circuit can completely stop when the biological sensor unit 100 does not drive, so that malfunction or failure can be inhibited from occurring. In addition, since the circuit can be brought into organification, a method of discarding the biological sensor unit 100 can be further simplified (a load to the environment due to the discard can be reduced). Furthermore, recycling of the biological sensor unit 100 can be achieved more easily.

In addition, the electric supply from the radio cooperative apparatus 200 to the biological sensor unit 100, is performed by the radio electric supply and furthermore the transmission of the biological information from the biological sensor unit 100 to the radio cooperative apparatus 200 is performed in the radio communication, and thus the radio cooperative apparatus 200 is required at least to be held over from above the clothes as in the example of B of FIG. 3. Therefore, there is no need for the user to take the clothes off in every piece of measurement, and thus the measurement can be performed more easily. With this arrangement, the sensor system (the biological sensor unit 100 and the radio cooperative apparatus 200) can be used more easily even in measurement in an able-bodied person's daily life. That is, the convenience of the sensor system (the biological sensor unit 100 and the radio cooperative apparatus 200) can improve.

In addition, since the radio cooperative apparatus 200 can supply the normal power larger than the power for driving the RFID tag, to the biological sensor unit 100, the biological sensor unit 100 can perform the sensing accompanied with a high degree of processing, such as the ultrasonic echo examination or the OCT examination.

<Flow of Detection Processing>

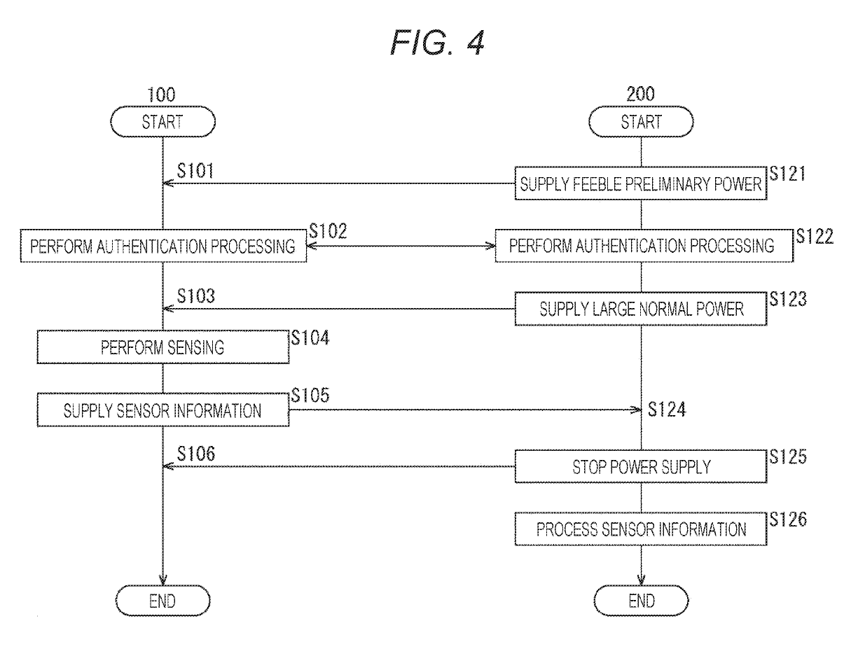

Next, the processing to be performed by the biological sensor unit 100 and the radio cooperative apparatus 200, will be described. An exemplary flow of detection processing of detecting the biological information will be described with reference to the flowchart of FIG. 4.

At step S121, the power source unit 211 of the radio cooperative apparatus 200 supplies (the radio electric supply) preliminary power being a degree of feeble power with which the authentication unit 112 can drive, to the biological sensor unit 100 through the antenna 213. At step S101, the electric supply reception unit 113 of the biological sensor unit 100 receives the preliminary power through the antenna 111. The electric supply reception unit 113 drives the authentication unit 112 with the preliminary power.

At step S122, the authentication unit 212 of the radio cooperative apparatus 200 performs the authentication processing in the radio communication with the biological sensor unit 100 (the authentication unit 112) through the antenna 213. At step S101, the authentication unit 112 of the biological sensor unit 100 performs the authentication processing in the radio communication with the radio cooperative apparatus 200 (the authentication unit 212) through the antenna 111.

When the authentication succeeds, at step S123, the power source unit 211 of the radio cooperative apparatus 200 starts supplying the normal power (the radio electric supply) to the biological sensor unit 100. At step S103, the electric supply reception unit 113 of the biological sensor unit 100 starts receiving the radio electric supply of the normal power. The electric supply reception unit 113 starts supplying the normal power to the sensor head 121 to the radio modulator 124. That is, the electric supply reception unit 113 drives the respective processing units of the sensor head 121 to the radio modulator 124, with the normal power.

At step S104, the sensor head 121 performs the sensing of the biological information. Then, at step S105, the amplifier 122 amplifies the sensor information (the biological information) acquired by the sensing. The A/D converter 123 performs the A/D conversion to the biological information. The radio modulator 124 supplies (transmits) the biological information as the radio signal to the radio cooperative apparatus 200 through the antenna 125.

At step S124, the radio demodulator 222 of the radio cooperative apparatus 200 receives the radio signal through the antenna 221.

With the reception of the radio signal, as step S125, the power source unit 211 of the radio cooperative apparatus 200 finishes the supply of the normal power (the radio electric supply) to the biological sensor unit 100. At step S106, the electric supply reception unit 113 of the biological sensor unit 100 finishes the reception of the normal power from the radio cooperative apparatus 200. That is, the electric supply reception unit 113 finishes the power supply to the sensor head 121 to the radio modulator 124.

At step S126, each of the processing units of the radio cooperative apparatus 200 performs the processing regarding the sensor information (the biological information) received at step S124. For example, the radio demodulator 222 demodulates the received signal to extract the biological information. The detecting demodulator 224 and the signal shaping unit 226 each perform the signal processing to the biological information. The analysis processing unit 228 performs the processing of, for example, analyzing the biological information.

When the processing to the sensor information finishes, the detection processing finishes.

Note that, in a case where the authentication processing at step S102 and the authentication processing at step S122 fail, the power source unit 211 stops (cancels) supplying the power (the radio electric supply) and the electric supply reception unit 113 prohibits (cancels) the acquisition of the power (the reception of the radio electric supply). Then, the detection processing finishes.

With the performance of the detection processing as described above, the convenience of the sensor system (the biological sensor unit 100 and the radio cooperative apparatus 200) can improve such that the miniaturization of the biological sensor unit 100 can be achieved more easily, and the like.

In addition, with the performance of the pieces of authentication processing as described above, information is inhibited from being transmitted or received between wrong devices and thus safer radio communication can be performed. Illegal information leaks, such as acquisition of the sensor information to be personal information with a biological sensor unit driven by a wrong radio cooperative apparatus, can be inhibited.

In addition, with the performance of the pieces of authentication processing in the supply of the power (the radio electric supply), the biological sensor unit 100 can be inhibited from driving unnecessarily. With this arrangement, for example, each of the processing units can be inhibited from being supplied with power when no sensor information is provided, the power being supplied by the wrong radio cooperative apparatus, and thus, for example, aging degradation, occurrence of malfunction, or unnecessary heating can be inhibited. That is, the reliability of the biological sensor unit 100 can improve. In addition, the radio cooperative apparatus 200 can inhibit the power supply to a wrong biological sensor unit 100, so that power consumption can be inhibited from increasing. For example, in a case where the radio cooperative apparatus 200 drives with a battery as the power source, the operating time of the radio cooperative apparatus 200 can further lengthen.

In addition, the radio cooperative apparatus 200 supplies the feeble preliminary power to the biological sensor unit 100, and supplies the normal power larger than the preliminary power in a case where the authentication succeeds, or cancels the supply of the power in a case where the authentication fails, so that unnecessary power can be inhibited from being supplied and the power consumption of the radio cooperative apparatus 200 can be inhibited from increasing. For example, in a case where the radio cooperative apparatus 200 drives with a battery as the power source, the operating time of the radio cooperative apparatus 200 can further lengthen. In addition, the biological sensor unit 100 can inhibit reception of unnecessary large power, can inhibit unnecessary heating, and can inhibit influence on the living body and the sensing.

In addition, as described above, after the biological sensor unit 100 transmits the biological information to the radio cooperative apparatus 200, the power supply is finished from the radio cooperative apparatus 200 to the biological sensor unit 100. Therefore, unnecessary power supply can be inhibited. With this arrangement, the power consumption of the radio cooperative apparatus 200 can be inhibited from increasing and the unnecessary heating can be inhibited in the biological sensor unit 100, so that the influence on the living body and the sensing can be inhibited.

<Flow of Authentication Processing>

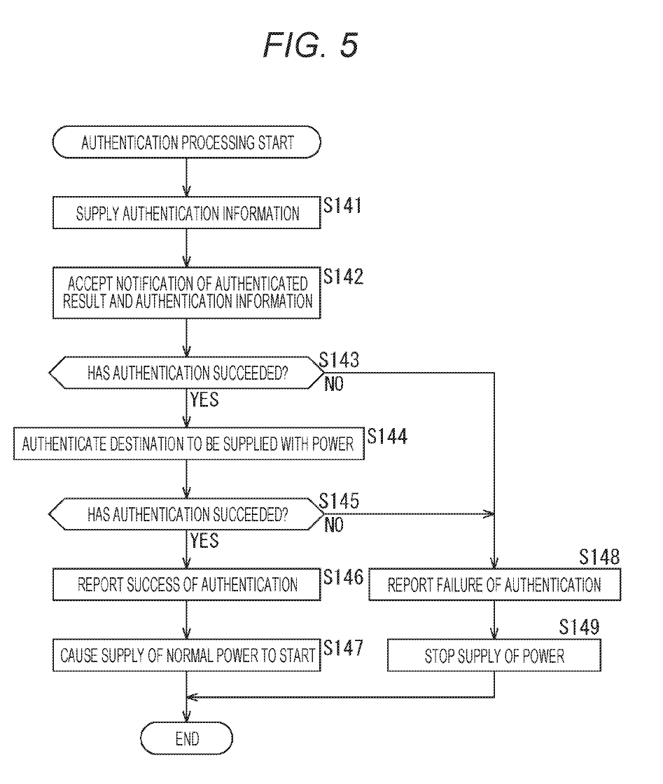

Next, an exemplary flow of the authentication processing to be performed by the radio cooperative apparatus 200 at step S122 (FIG. 4) will be described with reference to the flowchart of FIG. 5.

When the authentication processing starts, at step S141, the authentication unit 212 transmits (supplies) authentication information being information necessary for authenticating the radio cooperative apparatus 200, as a radio signal, to the biological sensor unit 100 through the antenna 213. The authentication information may include any information, and may include, for example, authentication information regarding hardware or software.

At step S142, the authentication unit 212 receives (accepts), through the antenna 213, a notification of an authenticated result to the authentication information transmitted at step S141 and authentication information regarding the biological sensor unit 100, transmitted as a radio signal from the biological sensor unit 100.

At step S143, the authentication unit 212 determines whether the biological sensor unit 100 has successfully authenticated the authentication information, on the basis of the notification accepted at step S142. In a case where it is determined that the authentication has succeeded, the processing proceeds to step S144.

At step S144, the authentication unit 212 authenticates the authentication information regarding the biological sensor unit 100 accepted at step S142, as a destination to be supplied with the power. At step S145, the authentication unit 212 determines whether the authentication has succeeded. In a case where it is determined that the success has been made, the processing proceeds to step S146.

At step S146, the authentication unit 212 notifies the biological sensor unit 100 of the effect that the authentication has succeeded, with a radio signal. In addition, at step S147, the authentication unit 212 causes the power source unit 211 to start supplying the normal power to the biological sensor unit 100 that has been authenticated.

When the processing at step S147 finishes, the authentication processing finishes and the processing goes back to FIG. 4. In addition, in a case where it is determined at step S143 or step S145 that the authentication has failed, the processing proceeds to step S148.

At step S148, the authentication unit 212 notifies the biological sensor unit 100 of the effect that the authentication has failed, with a radio signal. In addition, at step S149, the authentication unit 212 causes the power source unit 211 to stop supplying the normal power. When the processing at step S149 finishes, the authentication processing finishes and the processing goes back to FIG. 4.

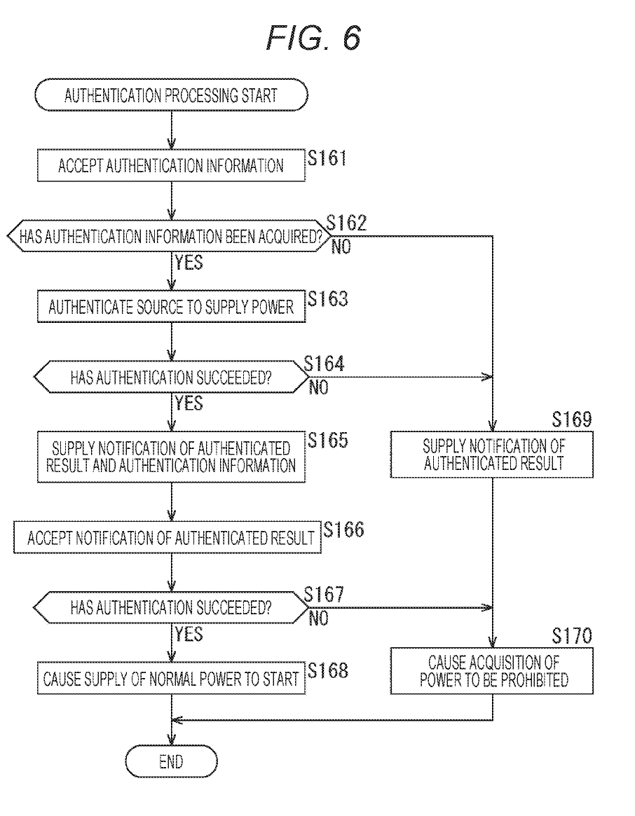

Next, an exemplary flow of the authentication processing to be performed by the biological sensor unit 100 at step S102 (FIG. 4) will be described with reference to the flowchart of FIG. 6.

When the authentication processing starts, at step S161, the authentication unit 112 receives (accepts) the authentication information regarding the radio cooperative apparatus 200 transmitted from the radio cooperative apparatus 200, as the radio signal, through the antenna 111.

At step S162, the authentication unit 112 determines whether the authentication information regarding the radio cooperative apparatus 200 has been acquired (received). In a case where the acquisition has been made, the processing proceeds to step S163.

At step S163, the authentication unit 112 authenticates the authentication information regarding the radio cooperative apparatus 200 accepted at step S161, as a source to supply the power. At step S164, the authentication unit 112 determines whether the authentication has succeeded. In a case where it is determined that the success has been made, the processing proceeds to step S165.

At step S165, the authentication unit 112 transmits (supplies) the notification of the authenticated result and the authentication information regarding the biological sensor unit 100, as the radio signal, to the radio cooperative apparatus 200 through the antenna 111.

At step S166, the authentication unit 112 receives (accepts) a notification of an authenticated result to the authentication information transmitted at step S165, transmitted as a radio signal from the radio cooperative apparatus 200, through the antenna 111.

At step S167, the authentication unit 112 determines whether the radio cooperative apparatus 200 has successfully authenticated the authentication information, on the basis of the notification accepted at step S166. In a case where it is determined that the authentication has succeeded, the processing proceeds to step S168.

At step S168, the authentication unit 112 causes the electric supply reception unit 113 to start acquiring the normal power from the radio cooperative apparatus 200 that has been authenticated.

When the processing at step S168 finishes, the authentication processing finishes and the processing goes back to FIG. 4. In addition, in a case where it is determined at step S162 or step S164 that the authentication has failed, the processing proceeds to step S169.

At step S169, the authentication unit 112 notifies the radio cooperative apparatus 200 of the effect that the authentication has failed, with a radio signal. When the processing at step S169 finishes, the processing proceeds to step S170. In addition, in a case where it is determined at step S167 that the authentication has failed, the processing proceeds to step S170.

At step S170, the authentication unit 112 causes the electric supply reception unit 113 to prohibit the acquisition of the power. When the processing at step S170 finishes, the authentication processing finishes and the processing goes back to FIG. 4.

In this manner, the biological sensor unit 100 and the radio cooperative apparatus 200 authenticate each other as the pieces of authentication processing. Therefore, not only the biological sensor unit 100 can inhibit power supply from a wrong radio cooperative apparatus 200, but also the radio cooperative apparatus 200 can be inhibited from supplying the power to a wrong biological sensor unit 100. Therefore, the security in the radio communication can further improve. Furthermore, for example, aging degradation, occurrence of malfunction, or unnecessary heating can be inhibited in the biological sensor unit 100, and additionally the power consumption of the radio cooperative apparatus 200 can be inhibited from increasing.

<Utilization of Biological Information>

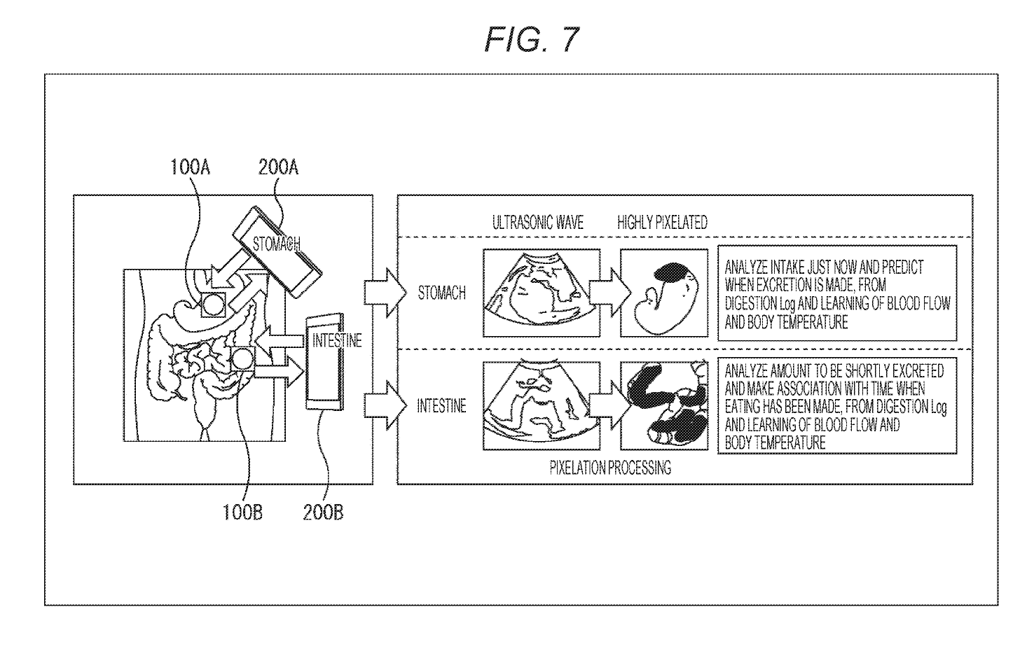

The method of using the biological information acquired as described above, is arbitrary. For example, as illustrated in FIG. 7, the biological sensor unit 100A applied to the skin of the surface region of a stomach, may perform imaging to the contents of the stomach with, for example, an ultrasonic sensor or an OCT sensor, and may wirelessly send the image signal to the radio cooperative apparatus 200A held over from above the clothes. In addition, the radio cooperative apparatus 200A may estimate the amount and types of the contents from an image of the contents of the stomach. In addition, the radio cooperative apparatus 200A may bring the image into high resolution with image processing to improve the estimate accuracy thereof.

For example, the user may hold the radio cooperative apparatus 200A over the biological sensor unit 100A from above the clothes after every meal, to estimate the amount and types of the contents of the stomach. The radio cooperative apparatus 200A may improve the accuracy of analyzing, for example, the nutrition content of an ingested meal, by machine learning on the basis of repetition of the estimation and if necessary collation with meal images or records separately acquired.

In addition, for example, as illustrated in FIG. 7, the biological sensor unit 100B applied to the skin of the surface region of the large intestine, may perform imaging to the contents of the large intestine with, for example, an ultrasonic sensor or an OCT sensor, and may wirelessly send the image signal to the radio cooperative apparatus 200B held over from above the clothes. In addition, the radio cooperative apparatus 200B may estimate the amount and types of the contents from an image of the contents of the large intestine. In addition, the radio cooperative apparatus 200B may bring the image into high resolution with image processing to improve the estimate accuracy thereof.

For example, the user may hold the radio cooperative apparatus 200B over the biological sensor unit 100B from above the clothes after every meal, to estimate the amount and types of the contents of the large intestine, and the radio cooperative apparatus 200B may improve the accuracy of analyzing, for example, the nutrition remaining amount of feces to be excreted, by machine learning on the basis of repetition of the estimation and if necessary collation with feces images or records separately acquired.

In addition, the biological sensor unit 100 may be further provided with, for example, sensors for temperatures, blood flows, pulse waves, pulses, and heartbeats, to transmit sensor information acquired from the sensors, as the biological information, to the radio cooperative apparatus 200. The radio cooperative apparatus 200 may perform the analysis processing with the sensor information to improve the accuracy of analyzing, for example, the nutrition content of an ingested meal or the nutrition remaining amount of feces to be excreted.

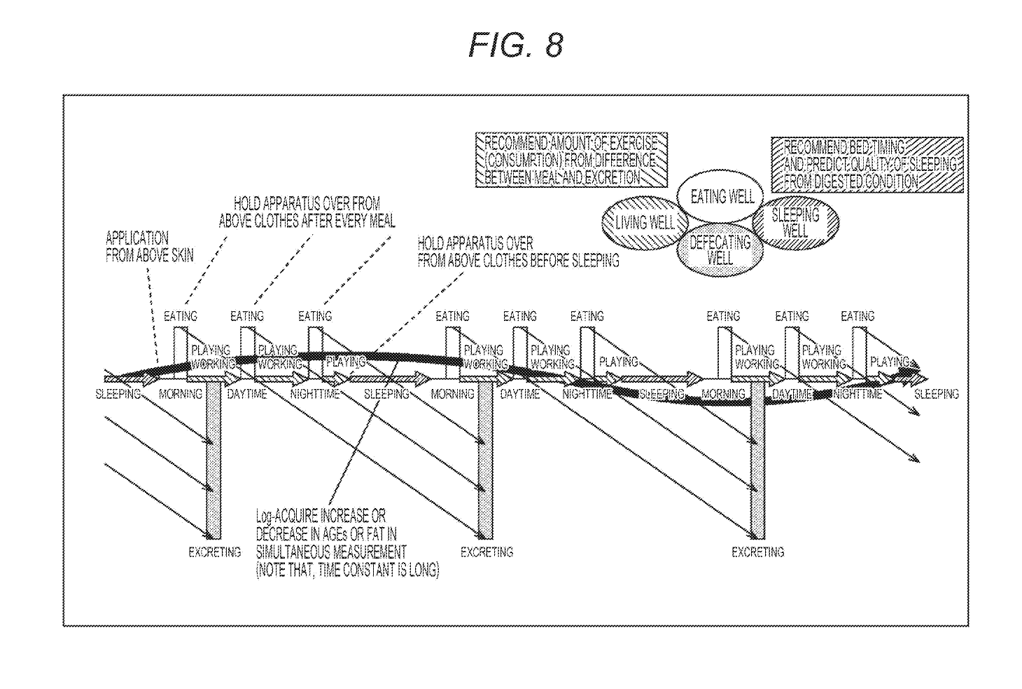

In addition, for example, as illustrated in FIG. 8, the radio cooperative apparatus 200 may analyze and display the causal relationship between when an ingested meal is to be excreted and when the ingestion of feces to be excreted has been made, on the basis of the accumulation of machine learning after every meal, and additionally may recommend exercise consumption for nutrition and living activities, on the basis of the difference between for example, the nutrition content of the ingested meal and the nutrition remaining amount of the feces to be excreted, for example, in order to make the difference zero.

For example, when the user holds the radio cooperative apparatus 200 over the biological sensor unit 100 from above the clothes before sleeping, the radio cooperative apparatus 200 may perform processing of, for example, recommending a bed timing optimal for sleeping well, on the basis of analysis of the digesting progress of the meal.

Furthermore, for example, the radio cooperative apparatus 200 may capture a separately acquired metabolic indicator, for example, a varied value in a body fat ratio or advanced glycation endproducts (AGEs) and may verify the effect of the recommendations for the exercise consumption for nutrition and for the living activities or the effect of the recommendation for the sleeping timing, to further improve the accuracy of recommendation by machine learning.

As described above, the radio cooperative apparatus 200 analyzes the biological information acquired from the biological sensor unit 100, to be able to perform the various types of processing.

The descriptions in which the biological sensor unit 100 detects the biological information from a living body, have been given above, but the object to be detected may be any object. Thus, the information regarding the object to be detected, is not limited to the biological information and the object to be detected is not limited to a living body. Furthermore, the present technology is not limited to the sensor unit and the radio cooperative apparatus described above, and thus can be applied to an arbitrary device. For example, the present technology can be applied to an arbitrary measurement device, image pickup device, communication device, power supply control device, information processing device, or electronic apparatus. That is, the present technology can be applied to a device or a system in an arbitrary field, such as transport, medical treatment, security, agriculture, livestock industries, mining industries, beauty care, factories, home electronics, weather, or nature monitoring.

<Software>

The set of processing described above can be performed by hardware or can be performed by software. In a case where the set of processing is performed by software, a program included in the software is installed into a computer. Examples of the computer include a computer built in dedicated hardware and a general-purpose personal computer in which various functions can be performed with installation of various programs.

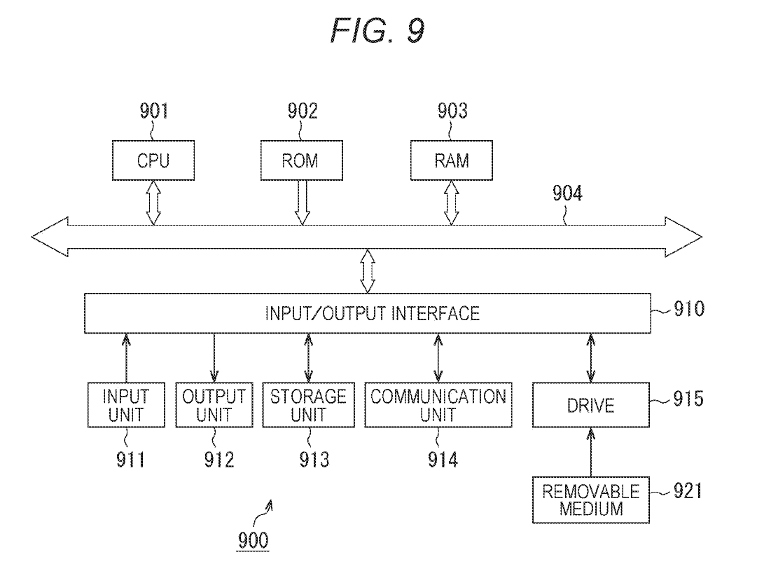

FIG. 9 is a block diagram illustrating an exemplary configuration of the hardware of the computer that performs the set of processing described above, with the program.

The computer 900 illustrated in FIG. 9 includes a central processing unit (CPU) 901, a read only memory (ROM) 902, and a random access memory (RAM) 903 mutually connected through a bus 904.

An input/output interface 910 is also connected with the bus 904. An input unit 911, an output unit 912, a storage unit 913, a communication unit 914, and a drive 915 are connected to the input/output interface 910.

Examples of the input unit 911 include a keyboard, a mouse, a microphone, a touch panel, and an input terminal. Examples of the output unit 912 include a display, a speaker, and an output terminal. Examples of the storage unit 913 include a hard disk, a RAM disk, and a non-volatile memory. The communication unit 914 includes, for example, a network interface. The drive 915 drives a removable medium 921, such as a magnetic disk, an optical disc, a magneto-optical disc, or a semiconductor memory.

In the computer 900 having the above configuration, for example, the CPU 901 loads the program stored in the storage unit 913 into the RAM 903 through the input/output interface 910 and the bus 904 and executes the program, so that the set of processing described above is performed. The RAM 903 appropriately stores, for example, data necessary for the CPU 901 to perform the various types of processing.

The program to be executed by the CPU 901 is, for example, recorded onto the removable medium 921 as a packaged medium, so that the program can be applied. In that case, the removable medium 921 is set into the drive 925, so that the program can be installed into the storage unit 913 through the input/output interface 910.

In addition, the program can be also supplied through a wired or radio transmission medium, such as a local area network, the Internet, or digital satellite broadcasting. In that case, the communication unit 914 receives the program, and then the program can be installed into the storage unit 913.

Alternatively, the program can be previously installed into the ROM 902 or the storage unit 913.

Note that the program to be executed by the computer may be a program for performing the processing on a time-series basis in the order described in the present specification, or may be a program for performing the processing in parallel or at a necessary timing at which a call is made or the like.

In addition, in the present specification, steps describing the program to be recorded in the recording medium, include not only the processing to be performed on the time-series basis in the order described but also the processing to be performed in parallel or individually even when the processing is not necessarily performed on the time series basis.

In addition, the processing at each step described above can be performed in each device described above or in an arbitrary device other than each device described above. In that case, the device that performs the processing is required at least to have the function (e.g., a functional block) described above necessary for performing the processing. In addition, information necessary for the processing is required at least to be transmitted to the device appropriately.

<Others>

In addition, in the present specification, a system means an aggregate including a plurality of constituent elements (e.g., a device and a module (a component)) regardless of whether all the constituent elements are included in the same casing. Therefore, a plurality of devices connected through a network, the devices each being housed in a different casing, and one device including a plurality of modules housed in one casing, both are involved in the system.

In addition, a configuration described above as one device (or one processing unit) may be divided to form a plurality of devices (or processing units). Conversely, configurations described above as a plurality of devices (or processing units) may be collectively formed to form one device (or one processing unit). In addition, a configuration other than the configurations described above may be added to the configuration of each device (or each processing unit). Furthermore, if the configuration or the operation of the entire system remains substantially the same, the configuration of a device (or a processing unit) may be partially included in the configuration of a different device (or a different processing unit).

The preferred embodiment of the present disclosure has been described above in detail with reference to the attached drawings, but the technical scope of the present disclosure is not limited to the example. It is obvious that a person skilled in the technical field of the present disclosure conceives various alterations or modifications in the scope of the technical idea described in the claims, and thus it is understood that these rightfully belong to the technical scope of the present disclosure.

For example, the present technology can have the configuration of cloud computing that performs processing with a plurality of devices cooperating with each other, the devices including one function divided through a network.

In addition, each step described in the flowcharts described above can be divided into a plurality of devices so as to be performed, in addition to being performed by one device.

Furthermore, in a case where one step includes a plurality of pieces of processing, the plurality of pieces of processing included in the one step can be divided into a plurality of devices so as to be performed, in addition to being performed by one device.

Note that, the embodiment of the present technology is not limited to the embodiment described above, and thus various alterations may be made without departing from the spirit of the present technology. For example, the present technology can be performed as not only a device or a system including the entirety or part of the configuration of the biological sensor unit 100 or the radio cooperative apparatus 200 described above, mounted, or a manufacturing device or manufacturing method thereof, but also any configuration including the entirety or part of the configuration of the biological sensor unit 100 or the radio cooperative apparatus 200 described above, mounted, for example, a processor as system large scale integration (LSI) and the like, a module including a plurality of processors and the like, a unit including a plurality of modules and the like, or a set including a different function further added to the unit, or a manufacturing device or manufacturing method for manufacturing the configuration.

Note that the present technology can also have the following configurations.

(1) An information processing device including:

an authentication unit configured to perform authentication processing with a different information processing device; and

a power supply unit configured to supply power to the different information processing device in a case where authentication of the authentication unit succeeds.

(2) The information processing device according to (1), in which the power supply unit supplies preliminary power for the authentication to the different information processing device before the authentication of the authentication unit,

the power supply unit being configured to start supplying normal power for driving the different information processing device in a normal state, to the different information processing device in the case where the authentication of the authentication unit succeeds, the normal power having power larger than the preliminary power,

the power supply unit being configured to stop supplying the power in a case where the authentication of the authentication unit fails.

(3) The information processing device according to (1) or (2), in which the power supply unit supplies the power to the different information processing device in radio communication.

(4) The information processing device according to any of (1) to (3), in which the authentication unit and the different information processing device authenticate each other.

(5) The information processing device according to any of (1) to (4), further including:

a reception unit configured to receive a transmitted signal transmitted by the different information processing device.

(6) The information processing device according to (5), in which the transmitted signal is a radio signal.

(7) The information processing device according to (5) or (6), in which the transmitted signal includes sensor information being information detected by a sensor included in the different information processing device.

(8) The information processing device according to (7), in which the power supply unit finishes the supply of the power after the reception unit receives the sensor information.

(9) The information processing device according to (7) or (8), further including:

an information processing unit configured to process the sensor information received by the reception unit.

(10) An information processing method including:

performing authentication processing with a different information processing device; and

supplying power to the different information processing device in a case where authentication succeeds.

(11) An information processing device including:

an authentication unit configured to perform authentication processing with a different information processing device; and

a power acquisition unit configured to acquire power supplied from the different information processing device in a case where authentication of the authentication unit succeeds.

(12) The information processing device according to (11), in which the power acquisition unit acquires the power supplied from the different information processing device in radio communication.

(13) The information processing device according to (11) or (12), in which the authentication unit and the different information processing device authenticate each other.

(14) The information processing device according to any of (11) to (13), in which the power acquisition unit acquires preliminary power for the authentication, supplied from the different information processing device before the authentication of the authentication unit,

the authentication unit performs the authentication processing with the preliminary power acquired by the power acquisition unit, and

the power acquisition unit starts acquiring normal power for driving the information processing device in a normal state, supplied from the different information processing device in the case where the authentication of the authentication unit succeeds, the normal power having power larger than the preliminary power, the power acquisition unit being configured to prohibit the acquisition of the power in a case where the authentication of the authentication unit fails.

(15) The information processing device according to any of (11) to (14), further including:

a transmission unit configured to transmit a transmitted signal to the different information processing device, the transmission unit being configured to drive with the normal power acquired by the power acquisition unit.

(16) The information processing device according to (15), in which the transmitted signal is a radio signal.

(17) The information processing device according to (15) or (16), further including:

a sensor unit configured to detect a predetermined parameter, the sensor unit being configured to drive with the normal power acquired by the power acquisition unit,

in which the transmission unit transmits sensor information being information regarding a detected result of the sensor unit, as the transmitted signal.

(18) The information processing device according to (17), in which the sensor unit detects biological information being information regarding a living body.

(19) The information processing device according to any of (15) to (18), in which the power acquisition unit finishes the acquisition of the normal power after the transmission unit finishes the transmission of the transmitted signal.

(20) An information processing method including:

performing authentication processing with a different information processing device; and

acquiring power supplied from the different information processing device in a case where authentication succeeds.

### REFERENCE SIGNS LIST

100 Biological sensor unit
111 Antenna
112 Authentication unit
113 Electric supply reception unit
121 Sensor head
122 Amplifier
123 A/D converter
124 Radio modulator
125 Antenna
200 Radio cooperative apparatus
211 Power source unit
212 Authentication unit
213 Antenna
221 Antenna
222 Radio demodulator
223 Memory
224 Detecting demodulator
225 Memory
226 Signal shaping unit
227 Memory
228 Analysis processing unit
231 Control unit
900 Computer

1. An information processing device comprising:
an authentication unit configured to perform authentication processing with a different information processing device; and
a power supply unit configured to supply power to the different information processing device in a case where authentication of the authentication unit succeeds.

2. The information processing device according to claim 1, wherein the power supply unit supplies preliminary power for the authentication to the different information processing device before the authentication of the authentication unit,
the power supply unit being configured to start supplying normal power for driving the different information processing device in a normal state, to the different information processing device in the case where the authentication of the authentication unit succeeds, the normal power having power larger than the preliminary power,
the power supply unit being configured to stop supplying the power in a case where the authentication of the authentication unit fails.

3. The information processing device according to claim 1, wherein the power supply unit supplies the power to the different information processing device in radio communication.

4. The information processing device according to claim 1, wherein the authentication unit and the different information processing device authenticate each other.

5. The information processing device according to claim 1, further comprising:
a reception unit configured to receive a transmitted signal transmitted by the different information processing device.

6. The information processing device according to claim 5, wherein the transmitted signal is a radio signal.

7. The information processing device according to claim 5, wherein the transmitted signal includes sensor information being information detected by a sensor included in the different information processing device.

8. The information processing device according to claim 7, wherein the power supply unit finishes the supply of the power after the reception unit receives the sensor information.

9. The information processing device according to claim 7, further comprising:
an information processing unit configured to process the sensor information received by the reception unit.

10. An information processing method comprising:
performing authentication processing with a different information processing device; and
supplying power to the different information processing device in a case where authentication succeeds.

11. An information processing device comprising:
an authentication unit configured to perform authentication processing with a different information processing device; and
a power acquisition unit configured to acquire power supplied from the different information processing device in a case where authentication of the authentication unit succeeds.

12. The information processing device according to claim 11, wherein the power acquisition unit acquires the power supplied from the different information processing device in radio communication.

13. The information processing device according to claim 11, wherein the authentication unit and the different information processing device authenticate each other.

14. The information processing device according to claim 11, wherein the power acquisition unit acquires preliminary power for the authentication, supplied from the different information processing device before the authentication of the authentication unit,
the authentication unit performs the authentication processing with the preliminary power acquired by the power acquisition unit, and
the power acquisition unit starts acquiring normal power for driving the information processing device in a normal state, supplied from the different information processing device in the case where the authentication of the authentication unit succeeds, the normal power having power larger than the preliminary power, the power acquisition unit being configured to prohibit the acquisition of the power in a case where the authentication of the authentication unit fails.

15. The information processing device according to claim 11, further comprising:
a transmission unit configured to transmit a transmitted signal to the different information processing device, the transmission unit being configured to drive with the normal power acquired by the power acquisition unit.

16. The information processing device according to claim 15, wherein the transmitted signal is a radio signal.

17. The information processing device according to claim 15, further comprising:
a sensor unit configured to detect a predetermined parameter, the sensor unit being configured to drive with the normal power acquired by the power acquisition unit,
wherein the transmission unit transmits sensor information being information regarding a detected result of the sensor unit, as the transmitted signal.

18. The information processing device according to claim 17, wherein the sensor unit detects biological information being information regarding a living body.

19. The information processing device according to claim 15, wherein the power acquisition unit finishes the acquisition of the normal power after the transmission unit finishes the transmission of the transmitted signal.

20. An information processing method comprising:
performing authentication processing with a different information processing device; and
acquiring power supplied from the different information processing device in a case where authentication succeeds.
